(19) United States
(12) Patent Application Publication
(10) Pub. No.: US 2006/0154299 A1
(43) Pub. Date: Jul. 13, 2006

(54) Protein microarray device having internal calibrators and methods of using therefor

(76) Inventors: Michael A. Harvey, Spofford, NH (US); Brett A. Stillman, Swanzey, NH (US); Thomas P. Owen, Swanzey, NH (US); Stephen A. Judice, Winchester, NH (US)

Correspondence Address: BRIAN D. VOYCE, 8401 STERLING BRIDGE ROAD, CHAPEL HILL, NC, 27516, US

(21) Appl. No.: 11/325,814

(22) Filed: Jan. 5, 2006

Related U.S. Application Data
(60) Provisional application No. 60/642,358, filed on Jan. 8, 2005.

(51) Int. Cl. G01N 33/53; C12M 1/34

(52) U.S. Cl. 435/7.1; 435/287.2

(57) ABSTRACT

The present invention relates to protein microarray devices having an internal calibrator, kits containing such devices, and methods of using such devices. Such a device comprises a plurality of protein arraying pads on a support substrate, a plurality of protein arraying spots on each pad, and a calibrator disposed on each pad, each calibrator comprising a series of spots of increasing concentration of a protein. In preferred embodiments, each pad has an immunoglobulin calibrator that is of the same species as the proteins that are reactive to the protein arraying spots. An advantage of the present invention is that one can use reliably use microporous surfaces for conducting multiplexed protein microimmunoassays with an assurance as to surface-to-surface fluorescence variability on a microarray device.

### BACKGROUND OF THE INVENTION

(1) Field of the Invention

The present invention relates to protein microarray devices having an internal calibrator, kits containing such devices, and methods of using such devices. Such a device comprises a plurality of protein arraying pads on a support substrate, a plurality of protein arraying spots on each pad, and a calibrator disposed on each pad, each calibrator comprising a series of spots of increasing concentration of a protein.

In preferred embodiments, each pad has an immunoglobulin calibrator that is of the same species as the antibody proteins that are reactive to the protein arraying spots. An advantage of the present invention is that one can use reliably use microporous surfaces for conducting quantitative multiplexed protein microimmunoassays with an assurance as to minimal surface-to-surface fluorescence variability on a microarray device.

(2) Description of the Related Art, Including Information Disclosed Under 37 CFR 1.97 & 1.98

DNA and protein microarrays have become important methods to allow the simultaneous interrogation of multiple binding reactions (cf. Schena, M. et al., Science 270:467-469(1995), Duggan, D. J., et al., Nature Genetics 21:10-14, (1999), MacBeath, G. and Schrieber, S. L. Science 289:1760-1763(2000). Testing multiple samples at the same time for binding activity against all the elements of a given array significantly increases the power of parallel processing with minimal sample volumes.

Protein (antigen) microarrays have been recognized as being useful for vaccine development and the serodiagnosis of infectious diseases. (See Bacarese-Hamilton T et alia, Biotechniques 33:S24-S29 (December 2002).) Arrays with controls and calibrators have been shown on glass slides, but the art has recognized that “the development, of protein arrays for research and clinical applications has lagged behind, because of the poor stability of proteins, complex coupling chemistries, high variability, and weak detection signals. High density protein arrays remain difficult to generate and validate for clinical use”. (See Bacarese et alia at S26).

### REFERENCE TO BENEFIT OF A RELATED PROVISIONAL APPLICATION

The present invention is entitled to and requests the benefit of the filing date of provisional application Ser. No. 60/642,358, filed Jan. 8, 2005. This provisional application bears the same title as above and has been filed in the name of the same inventors.

### BRIEF SUMMARY OF THE INVENTION

Methods and devices to provide and improve high throughput analysis of biomolecules (nucleic acids, proteins etc.) are important for elucidation of protein function, diagnostic testing, drug discovery, and drug target identification. One set of technologies that have improved the simultaneous interrogation of large numbers of biomolecules is the use of microarrays. Microarrays are ordered displays of molecules generally immobilized on a surface. Such an array permits the simultaneous investigation of binding of many elements to target molecules. A variety of technologies have been developed to allow investigators to make, process and detect reactions on microarrays.

An object of the invention is to provide an addressable fluorescent protein microarray with multiple surfaces that can bind many different proteins, maintains the protein three dimensional structure, immobilize them in sufficient quantity to allow for sensitive, rapid detection, and allow for surface-to-surface variability on each binding surface.

A second object of the invention is to be able to interrogate sensitively the same array of proteins with different samples or binding partners. Because specific protein binding partners are generally rare, any technique which allows the use of minimum quantities is preferred.

A third object of the invention is to use a convenient set of methodologies to allow high throughput techniques. In particular, an object of the invention is to use the “micro plate” 96 well format, which is based on 9 mm spacings of reaction areas which are 7 mm either in diameter or square. Many pipetting aids, detection instrumentation, liquid handling systems and robotics have been designed to conform to this format.

A fourth object of the present invention is to provide for parallel processing of substantially identical microarrays on multiple samples.

A fifth object of the present invention is to provide a method for creating standard curves for each binding surface.

A sixth object of the invention is to provide for simultaneously determining the concentration of different isotypes of specific binding partners, such as antibodies, simultaneously.

The present invention is a device for preparing multiple assay samples for multiple reactive sites located on at least one assay slide. Each slide comprises at least three elements—a plurality of protein arraying pads on a support substrate, a plurality of protein arraying spots on each pad, and a calibrator disposed on each pad. Each calibrator comprises a series of spots of increasing concentration of a protein.

Typically, each pad comprises a protein binding, three-dimensional, microporous surface for disposing high protein capacity per area. The support is a planar shaped glass slide having a set of exterior edges and a planar surface covered with a plurality of separate and discretely spaced assay reaction surface locations.

For the purposes of the present invention, the term “pad” refers to a surface area on the support that is capable of binding proteins at a plurality of areas, but is physically delimited and distinct from other such surface areas on the support. Thus, a support has multiples pads, each pad containing multiple protein arraying spots. Also, for the purposes of the present invention, “protein arraying spots” refers to distinct and delimited areas on each pad wherein a protein is applied or disposed on each pad. At least two spots must be on each pad, but there at least dozens if not hundreds.

Along with the support slides, the present invention may comprise at least one multiwell chamber plates. Each chamber plate has a plurality of bottomless wells located between a top planar surface and a bottom planar surface and encompassed by a set of exterior wall surfaces. Each chamber plate is dimensioned and configured so as to register the wells with the assay reaction surface locations of a corresponding assay slide. Each chamber plate is located adjacent to and in registration with the corresponding assay slide. Each chamber plate well is dimensioned so as to encompass the area of a corresponding assay reaction surface location on the corresponding assay slide. Each well is discrete from the other and is dimensioned so as to receive a sample. Each well has an opening that can communicate with the corresponding assay reaction surface location. Such chambers are commercially available from Whatman Schleicher & Schuell, located in Keene, N.H.

The calibrator present on each pad can comprise a series of deposited immunoglobulin spots, typically selected from a species of antibodies reactive with the deposited antigens, which in turn are the protein arraying spots. Each antigen is deposited on a different spot. In selecting the deposited immunoglobulin and the concentrations to be used (which vary in amount from spot to spot), one would keep in mind that the immunoglobulin is used to bind a fluorescently labeled specific binding partner to the immunoglobulin so as to be useful for generating a standard curve for the amount of immunoglobulin that binds to the deposited antigen on each protein arraying spot. Thus, with the calibrator series concentrations being known, the calibrator produces a series of detectable fluorescent spots of variable intensity related to the concentration of the disposed calibrator proteins when reacted with the fluorescently labeled specific binding partner.

The present invention also comprises the methods for using the above devices. One method for using such devices comprises detecting antibodies in a sample. A sample is aliquoted onto at least one protein arraying spot of a protein microarray comprised of a plurality of protein arraying pads on a support substrate, a plurality of protein arraying spots on each pad, and a calibrator disposed on each pad, each calibrator comprising a series of spots of known increasing concentration of a protein. Using conventional parameters, one reacts a sample with the protein arraying spots.

Each of the calibrator spots and the protein arraying spots is contacted with a fluorescently labeled specific binding partner applied across the pads. The fluorescent signal from each calibrator spot is read by conventional measuring equipment so as to generate a standard curve. Also, the fluorescent signal from each protein arraying spot is read to see interpret the presence and/or amount of protein bound from the sample.

Alternatively, one can place a number of samples onto any of a number of the pads. This flexibility allows the user to provide for a device that either can query as to one protein analyte in a number of samples (each spot containing the same protein binding partner and a different sample applied to each spot), can query as to a variety of proteins in one sample (each spot containing a different protein binding partner and an aliquot of the sample applied to each spot), or query as to a combination of both samples and protein analytes (the spot containing multiple examples of different protein binding partner and different sample aliquots applied to each series of similar spots).

Suitable samples include proteomic samples such as is selected from the group consisting of cell lysates, cell supernatants, plasma, serum, or other biological fluids.

### BRIEF DESCRIPTION OF THE DRAWINGS

FIG. 1 is a perspective view of the present invention showing the slide support and pads, as well as optional chambers.

FIG. 2 is a view of one of the pads of a present invention microarray device, showing the arrangement of protein arraying spots and calibrator spots.

FIG. 3 is a graph showing the linearity of signal from each of sixteen pads on the same support slide.

FIG. 4 is graph slide-to-slide reproducibility with specific array intensities.

FIG. 5 is graph slide-to-slide reproducibility with “IgG” units derived from pad calibrators.

### DETAILED DESCRIPTION OF THE INVENTION

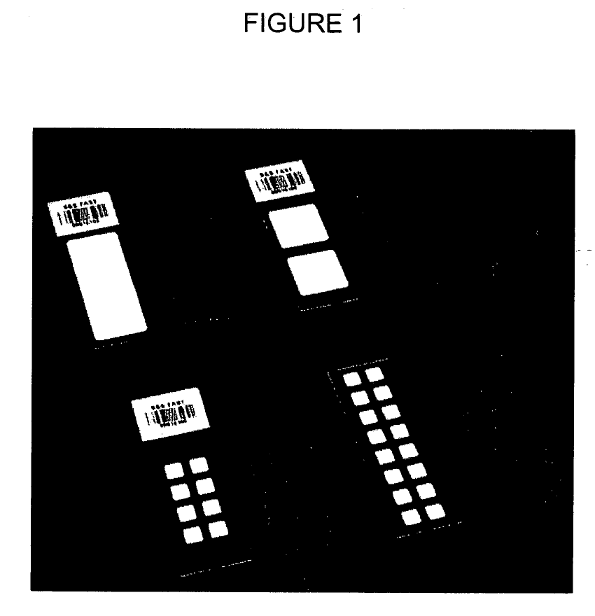

In preferred embodiments, the present invention relates to a device for preparing multiple assay samples. A preferred embodiment is shown in FIG. 1. The device comprises a standard-sized glass microscope slide (75 mm by 125 mm), comprising a planar support having a set of exterior edges and a planar surface covered with sixteen separate and discretely spaced pads. Each pad has a number of protein arraying spots deposited thereon in distinct patterns. The pads are present at a density and spacing that is registerable with that of a standard 96 well microplate or other conventional microplate formats such as 24 wells, 64 wells, and 384 wells. The device can comprise a series (4) of slides held in a holder sized to the dimensions of an SBS standard multiwell microplate, in this illustration a 96 well microplate having a 9 mm spacing format.

The pads can be constructed of a protein binding material that is disposed on the support slide. Suitable materials include microporous surface materials such as nitrocellulose, nylon, or polyvinyldifluoridene. Preferably the microporous surface is less than 15 um thick and greater than 2 um thick. The microporous surface can have pores of less than about 10 microns, preferably between about 10 microns and 0.05 microns. The protein binding capacity of the microporous surface can at least about 1 ug/mm³, preferably at least about 10 ug/mm³.

The protein arraying spots can be arranged on each pad using conventional printing means in regular patterns with a uniform centered spacing, typically using solutions of about 0.5 mg/ml of protein. Each spot can have a diameter of between about 50 um and about 1000 um. Calibrator spots can be in series of five variable known concentrations on each pad.

A high protein binding capacity combination of pads and spots as described above can provide greater speed, sensitivity, and dynamic range in a protein microarray.

A second preferred embodiment can include a device that can detect protein isotypes, such as immunoglobulin isotypes. The spots would comprise a protein capable of binding isotypes. Thus, in this embodiment, the calibrator must be capable of addressing any of the bound isotypes in a separate standard curve. The calibrator can be of two different configurations, either at least two series of spots, for example, each series having a different immunoglobulin isotype, or a series of spots, for example, each spot having at least two different immunoglobulin isotypes disposed thereon. One would use different fluors to detect the isotypes. For example, one can use Cye 3 coupled detector antibody and Cye 5 coupled detector antibody simultaneously.

Kits can be made that incorporate the above protein microarray devices along with any combination of associated equipment or reagents including slide holders, slide chambers, fluorescent binding reagents, or informatic software for generating standard curves and interpretative reading results of the microarray on the device.

The present invention can be used to detect the presence of autoreactive antibodies in a patient having an autoimmune disease, antibodies to viral diseases, antibodies to bacterial diseases, or or antibodies to allergic reactions

The present invention can be used for multiplexed infectious disease testing against hepatitus A/B/C, Epstein-Barr virus, human papilloma virus, Lyme Disease and others.

### EXAMPLE OF DEVICE USE

A device was made to incorporate a calibrator on each microarray pad so as to produce a standard curve that permits quantification of bound auto-antibodies to a series of twenty arrayed antigens. Each antigen was to be able to be quantified independent of other antigens.

Sixteen pad FAST brand slides (made by Whatman Schleicher and Schuell of Keene, N.H., USA) were arrayed with commercially available whole human IgG

A number of commercially available human immunoglobulin antigens were arrayed on the pad at the protein arraying spots using a Perkin Elmer BioChip Arrayer (of Boston, Mass., USA), see Table 1. Each pad had a different antigen placed on a pad. Each pad had a human IgG calibrator comprising a series of five concentrations ranging as follows—0.031 mg/ml, 0.062 mg/ml, 0.125 mg/ml, 0.25 mg/ml, and 0.5 mg/ml.

TABLE 1

| Autoantigen | |
|---|---|
| Ab | Clinical significance |
| dsDNA | Specificity of 90% for systemic lupus erythematodes |
| U170k | 70 kDa protein which is one component of the U-1 small |
| | nuclear ribonucleotide particle (snRNP) (Antibodies are |
| | associated with mixed connective tissue disease.) |
| SMAG | 29 kDa protein associated with snRNP (Antibodies are |
| | associated with systemic lupus erythematodes (SLE).) |
| SS-A/Ro52 | 52 kDa DNA binding protein (Antibodies are associated |
| | with neonatal lupus erythematodes (NEL) and SLE.) |
| SS-A/Ro60 | 60 kDA protein involved in the translation of ribosomal |
| | proteins (Antibodies are associated with NLE.) |
| SS-B/La | 48 kDA phosphoprotein representing a transcription factor |
| | for RNA polymerase III (Antibodies associated with |
| | Sjögren's syndrome, SLE, and NLE.) |
| Scl/Topo | 100 kDa helicase (Antibodies associated with sclerdoma or |
| | systemic sclerosis.) |
| PM-Scl/75 | 75 kDa nucleolar protein (Antibodies associated with |
| | polymyositis and systemic sclerosis.) |
| PM-Scl/100 | 100 kDa nucleolar protein (Antibodies associated with |
| | polymyositis and systemic sclerosis.) |
| Jo1 | Histidyl RNA synthetase (Antibodies detectable in |
| | polymyositis and systemic sclerosis.) |
| Pr3 | 30 kDa serin proteinase found in azurophilic granules of |
| | neutrophil granulocytes. |
| MPO | 59 kDA protein, a myeloperoxidase found in azurophilic |
| | granules of neutrophil granulocytes. |
| Mi-2 | 235-240 kDa protein (Antibodies associated with |
| | dermamyositis and polymyositis.) |
| GLOB | Proteins associasted with the glomerulur basement |
| | membrane (Antibodies associated with |
| | Goodpasture's syndrome.) |
| CENP-B | 80 kDA protein involved with maintaining chromatin |
| | structure within the centromere (Antibodies associated with |
| | sclerdoma variant.) |

In use, the microarrays were blocked for fifteen minutes using S&S Protein Array Blocking Buffer (made by Whatman Schleicher and Schuell of Keene, N.H., USA). Serum samples were added to each pad at a 1:100 dilution for one hour at room temperature. The serum was aspirated from the pads, followed by the pads being washed three times with TBS-T (a solution of 1×Tris buffered saline (TBS) and 2% Tween20 surfactant). The microarray pads were developed for a readout by incubating each pad with a commercially available fluorescently labeled secondary antibody (anti-human IgG coupled to Cy5 dye from Amersham Life Sciences) for one hour, approximately (a 1:8000 dilution of a 1 mg/ml stock) was added to each pad at 70 ul/pad. Again the pads were each washed with three rinses of TBS-T. The pads were air-dried and imaged in a fluorescent scanner, namely, an Axon Model 4200A scanner at laser settings of 75% and 330 PMT (made by Axon Instruments of Union City, Calif. USA). Spot finding and analysis were performed with Array Pro software (from Media Cybernetics of Silver Spring, Md. USA).

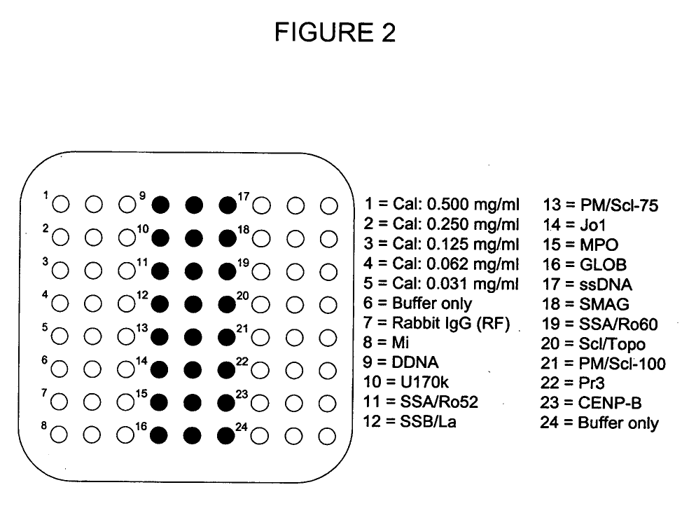

FIG. 2 is representative of the auto-antigen proteins arrayed on a pad of the present invention. The referenced proteins can be found in Table 1.

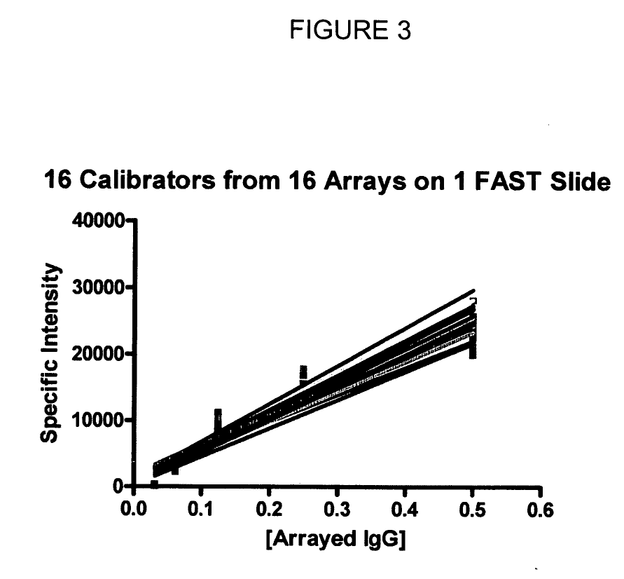

FIG. 3 shows a calibration curve developed from the IgG spots on each pad (16 calibrators from 16 pads on one slide support). Linear regression was used to generate a straight line representing the relationship between arrayed IgG and specific fluorescent intensity one each pad. Using these lines, specific intensity values from each auto-antigen arrayed element was interpolated to determine “IgG” units. These units were then used to characterize patient sera.

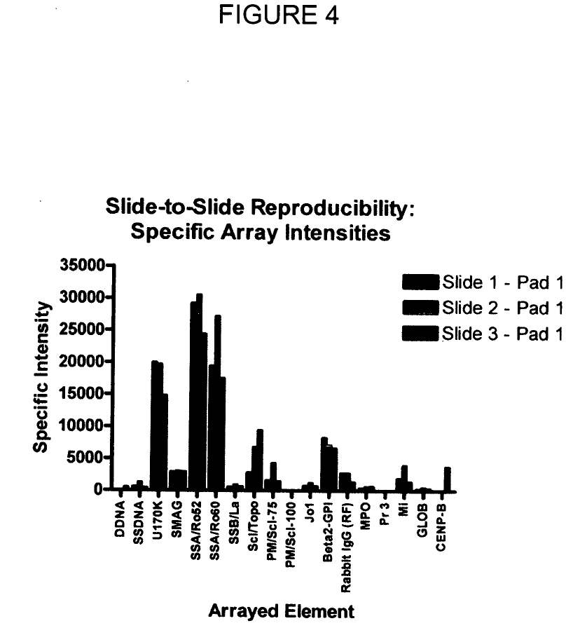

FIG. 4 illustrates slide-to-slide reproducibility of arrayed auto-antigens.

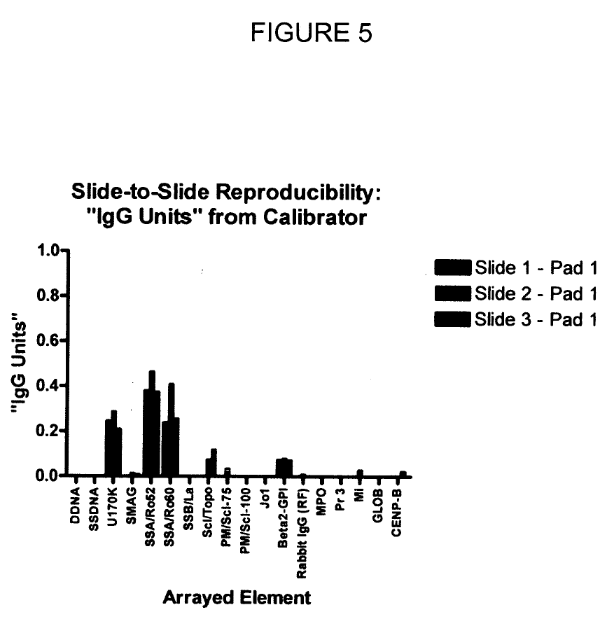

FIG. 5 illustrates slide-to-slide reproducibility using IgG units from the calibrators.

As shown in the Figures, the results demonstrate that the present device allows for the simultaneous processing of multiple samples on multiple arrays with internal calibrators for observing any pad-to-pad or slide-to-slide variability. Fluorescently labeled antibodies have been used to interrogate treated or untreated serum samples for the presence or amount of auto-antibodies.

The ordinarily skilled artisan can appreciate that the present invention can incorporate any number of the preferred features described above.

All publications or unpublished patent applications mentioned herein are hereby incorporated by reference thereto.

Other embodiments of the present invention are not presented here which are obvious to those of ordinary skill in the art, now or during the term of any patent issuing from this patent specification, and thus, are within the spirit and scope of the present invention.

We claim:

1. A protein microarray comprising:
a) a plurality of protein arraying pads on a support substrate;
b) a plurality of protein arraying spots on each pad; and
c) a calibrator disposed on each pad, each calibrator comprising a series of spots of increasing concentration of a protein.

2. The protein microarray of claim 1 wherein each pad comprises a protein binding, microporous surface.

3. The protein microarray of claim 2 wherein the microporous surface has pores of less than about 10 microns.

4. The protein microarray of claim 3 wherein the microporous surface has pores of between about 10 microns and 0.05 microns.

5. The protein microarray of claim 2 wherein the microporous surface is less than 15 um thick and greater than 2 um thick.

6. The protein microarray of claim 2 wherein the microporous surface is selected from the group consisting of nitrocellulose, nylon, or polyvinyldifluoridene.

7. The protein microarray of claim 2 wherein the microporous surface has a protein binding capacity of at least about 1 ug/mm³.

8. The protein microarray of claim 7 wherein the microporous surface has a protein binding capacity of at least about 10 ug/mm³.

9. The protein microarray of claim 1 wherein the spots are arranged with a uniform centered spacing.

10. The protein microarray of claim 1 wherein each spot has a diameter of between about 50 um and about 1000 um.

11. The protein microarray of claim 9 wherein each pad is dimensioned and configured to fit the well spacing in conventional multiwell test devices.

12. The protein microarray of claim 1 wherein the calibrator spots have a deposited immunoglobulin, selected from a species of antibodies reactive with the deposited antigens, and the protein arraying spots have at least two deposited antigens, each antigen being deposited on a different spot.

13. The protein microarray of claim 12 wherein the calibrator spots can be reacted with a fluorescently labeled specific binding partner so as to be useful for generating a standard curve for the amount of immunoglobulin that binds to the deposited antigen on each protein arraying spot.

14. The protein microarray of claim 1 wherein the calibrator series concentrations are known, and the calibrator produces a series of detectable fluorescent spots of variable intensity related to the concentration of the disposed calibrator proteins when reacted with a fluorescently labeled specific binding partner.

15. The protein microarray of claim 1 wherein the calibrator comprises at least two series of spots, each series having a different immunoglobulin isotype.

16. The protein microarray of claim 1 wherein the calibrator comprises at least two series of spots, each series having a different immunoglobulin isotype, the calibrator series concentrations are known, and the calibrator produces a series of detectable fluorescent spots of variable intensity related to the concentration of the calibrator when reacted with a fluorescently labeled specific binding partner.

17. The protein microarray of claim 1 wherein the calibrator comprises a series of spots, each spot having at least two different immunoglobulin isotypes disposed thereon.

18. The protein microarray of claim 1 wherein the calibrator comprises a series of spots, each spot having at least two different immunoglobulin isotypes disposed thereon, the calibrator series concentrations of the isotypes are known, and each calibrator isotype series produces a respective series of detectable fluorescent spots of variable intensity related to the concentration of each respective isotype when reacted with a respective differential fluorescently labeled specific binding partner.

19. A protein microarray comprising:
a) a plurality of protein arraying pads on a support substrate;
b) a plurality of protein arraying spots on each pad;
c) a calibrator disposed on each pad, each calibrator comprising a series of spots of increasing concentration of a protein;
d) at least one sample specific binding partner bound to at least one of the protein arraying spots; and
e) a fluorescently labeled specific binding partner bound to the calibrator spots and to the sample specific binding partners.

20. The protein microarray of claim 19 wherein the calibrator spots have a deposited immunoglobulin, selected from a species of antibodies reactive with the deposited antigens, and the protein arraying spots have at least two deposited antigens, each antigen being deposited on a different spot.

21. The protein microarray of claim 19 wherein the calibrator comprises a series of spots, each spot having at least two different immunoglobulin isotypes disposed thereon.

22. The protein microarray of claim 19 wherein the calibrator comprises at least two series of spots, each series having a different immunoglobulin isotype, the calibrator series concentrations are known, and the calibrator produces a series of detectable fluorescent spots of variable intensity related to the concentration of the calibrator when reacted with the fluorescently labeled specific binding partner.

23. The protein microarray of claim 19 wherein the calibrator comprises a series of spots, each spot having at least two different immunoglobulin isotypes disposed thereon.

24. The protein microarray of claim 19 wherein the calibrator comprises a series of spots, each spot having at least two different immunoglobulin isotypes disposed thereon, the calibrator series concentrations of the isotypes are known, and each calibrator isotype series produces a respective series of detectable fluorescent spots of variable intensity related to the concentration of each respective isotype when reacted with a respective differential fluorescently labeled specific binding partner.

25. A method for detecting antibodies in a sample comprising:
a) aliquoting a sample onto at least one protein arraying spot of a protein microarray comprised of:
i) a plurality of protein arraying pads on a support substrate;
ii) a plurality of protein arraying spots on each pad; and
iii) a calibrator disposed on each pad, each calibrator comprising a series of spots of increasing concentration of a protein;
b) contacting each of the calibrator spots and the protein arraying spots with a fluorescently labeled specific binding partner;
d) reading the fluorescent signal from each calibrator spot so as to generate a standard curve; and
e) reading the fluorescent signal from each protein arraying spot.

26. A method for detecting antibodies in a sample comprising:
a) aliquoting at least two samples onto respectively different protein arraying spots on a protein microarray comprised of:
i) a plurality of protein arraying pads on a support substrate;
ii) a plurality of protein arraying spots on each pad; and
iii) a calibrator disposed on each pad, each calibrator comprising a series of spots of increasing concentration of a protein;
b) contacting each of the calibrator spots and the protein arraying spots with a fluorescently labeled specific binding partner;
c) reading the fluorescent signal from each calibrator spot so as to generate a standard curve; and
f) reading the fluorescent signal from each protein arraying spot.

27. A kit for detecting multiplexed bound proteins comprising:
a) a protein microarray comprised of:
i) a plurality of protein arraying pads on a support substrate;
ii) a plurality of protein arraying spots on each pad; and
iii) a calibrator disposed on each pad, each calibrator comprising a series of spots of increasing concentration of a protein; and
b) a multiplexing assay component selected from the group consisting of pad chambers, specific binding detection reagents, and informatic fluorescent signal software.

28. A method of detecting the presence of an antibody to an auto-antigen comprising contacting a sample with a microarray comprising:
a) aliquoting a sample onto at least one protein arraying spot of a protein microarray comprised of:
iv) a plurality of protein arraying pads on a support substrate;
v) a plurality of protein arraying spots on each pad with a selection of auto-antigens each arrayed one to a spot; and
vi) a calibrator disposed on each pad, each calibrator comprising a series of spots of increasing concentration of a deposited immunoglobulin, selected from a species of antibodies reactive with the arrayed auto-antigens;
b) contacting each of the calibrator spots and the protein arraying spots with a fluorescently labeled specific binding partner;
g) reading the fluorescent signal from each calibrator spot so as to generate a standard curve; and
h) reading the fluorescent signal from each protein arraying spot so as to detect the presence of any auto-antigen antibodies.
